(19) United States
(12) Patent Application Publication
(10) Pub. No.: US 2026/0060828 A1
(43) Pub. Date: Mar. 5, 2026

(54) MEDICAL DEVICE RELOADING SYSTEM

(71) Applicant: Bolton Medical, Inc., Sunrise, FL (US)

(72) Inventors: Scott Lyle Rush, Ft. Lauderdale, FL (US); Timothy Lostetter, Cooper City, FL (US); Flavio Magistrelli, Sunrise, FL (US); Casey Torrance, Snohomish, WA (US)

(21) Appl. No.: 18/821,650

(22) Filed: Aug. 30, 2024

(51) Int. Cl. A61F 2/966; A61F 2/95

(52) U.S. Cl. CPC A61F 2/966; A61F 2002/9534

(57) ABSTRACT

Systems and methods for reloading a stent graft delivery device are disclosed. Additionally, a kit including a patient-specific fenestration template, fenestration rings and sutures can be provided for forming fenestrations in a preexisting, or generic, stent graft with the fenestrated stent graft then reloaded within the sheath of the delivery device for deployment within a patient.

### TECHNICAL FIELD

The disclosure is generally directed to the use of stent grafts, and in particular, modifying a partially deployed graft (e.g. to add patient-specific fenestrations) and reinserting the modified graft into the deployment device for delivery within a patient. For purpose of illustration and not limitation, an exemplary embodiment includes an expandable stent graft (though other grafts, e.g. without self-expanding stent struts, are within the scope of the present disclosure).

### BACKGROUND OF THE DISCLOSURE

Aneurysms generally involve the abnormal swelling or dilation of a blood vessel such as an artery. The wall of the abnormally dilated blood vessel is typically weakened and susceptible to rupture. For example, an abdominal aortic aneurysm (AAA) is a common type of aneurysm that poses a serious health threat. A common way to treat AAA and other types of aneurysm is to place an endovascular stent graft such that the stent graft spans across and extends beyond the proximal and distal ends of the diseased portion of the vasculature. The stent graft is designed to reline the diseased vasculature, providing an alternate blood conduit that isolates the aneurysm from the high pressure flow of blood, thereby reducing or eliminating the risk of rupture. The embodiments disclosed herein are particularly suited for treatment of aortic aneurysms that encompass or affect the visceral segment of the aorta.

Minimally invasive endovascular repair using stent grafts is often preferred to avoid the risks associated with traditional open surgical repair. However, these stent grafts can only be used when the graft can be placed in a stable position without covering major branch vessels. In the cases of juxtarenal aneurysm where the dilation extends up to but does not involve the renal arteries, the proximal portion of the stent graft needs to be secured to the aortic wall above the renal arteries, thereby blocking the openings to the renal arteries. Thus, patients with juxtarenal aneurysms, which represent a significant proportion of abdominal aortic aneurysm cases, are typically excluded from standard endovascular treatment.

To allow for endovascular repair of a wider range of cases, surgeons sometimes cut openings in the stent graft body to accommodate specific branch vessel origins, a process known as “fenestrating”. Thus, for example, in treating juxtarenal aneurysms, the fenestrations or openings of the stent grafts are to be aligned with the renal arteries. Traditionally, the fenestration process involves measurements based on medical images (such as CT scans) of the vessel origins. Longitudinal distances may be measured, and relative angular locations may be estimated from a reference point.

Stent grafts are manufactured such that the implantable device is loaded into a delivery sheath, which enables the physician to deploy the device into the anatomy with minimal surgical intervention. The delivery sheath is tracked through a patient's vasculature through a peripheral artery, commonly originating at the femoral artery. In cases where a physician wants to add a fenestration to a stent-graft after the device has been fully manufactured by the original manufacturer, the physician will need to take the stent-graft out of the delivery sheath, place the fenestrations, and then put the stent-graft back into the delivery sheath in order to deploy the device in the target anatomy while still using minimal surgical intervention. Accordingly, there is a need for a stent graft reloading method and kit to allow the physician to properly place the fenestrations, ensure that the fenestrations are appropriate for their intended clinical application and return the stent-graft to its original primary sheath.

### SUMMARY

According to certain aspects of the present disclosure, systems and methods are disclosed for applying modifications (e.g. fenestrations via a template with patient-specific fenestration locations) to a generic stent graft, and tooling to facilitate a rapid and consistent reloading process to the modified stent graft into the delivery system.

In one aspect of the disclosure, a method for reloading a graft into a graft delivery device, the graft being at least partially expanded and at least partially deployed from the graft delivery device, the method comprising: advancing a compressing device to at least partially compress the at least partially deployed graft; causing the at least partially compressed graft to be contained within the graft delivery device; and removing the compressing device.

In some embodiments, the method includes forming at least one fenestration in the graft. In some embodiments, forming the at least one fenestration includes causing the graft to be in an expanded configuration, and disposing a fenestration template over at least a portion of the expanded graft. In some embodiments, the template defines at least one patient-specific feature. In some embodiments, forming the at least one fenestration includes cauterizing a portion of the graft. In some embodiments, advancing the compressing device includes advancing a funnel in a proximal direction over the partially deployed fenestrated graft. In some embodiments, causing the at least partially compressed graft to be contained includes advancing a sheath of the graft delivery device in a distal direction over the compressed fenestrated graft.

In some embodiments, the method includes coupling a fenestration ring to the fenestrated graft, the fenestration ring being complementarily sized and shaped with the at least one fenestration. In some embodiments, coupling the fenestration ring to the fenestrated graft includes at least one suture. In some embodiments, the at least one patient-specific feature is determined from at least one scan of a patient's anatomy. In some embodiments, the at least one patient-specific feature includes a hole complementary with a feature of the patient's anatomy. In some embodiments, advancing the sheath of the delivery device in a distal direction over the compressed fenestrated graft includes advancing the sheath though the funnel. In some embodiments, the funnel has a frustoconical proximal portion and elongated cylindrical tube portion extending therefrom. In some embodiments, the funnel is advanced to dispose the fenestrated graft within the elongated cylindrical tube portion of the funnel.

In some embodiments, advancing a funnel over the at least partially deployed fenestrated graft includes: advancing a first funnel having a first diameter over the deployed fenestrated graft; and advancing a second funnel having a smaller diameter than the first funnel over the deployed fenestrated graft. In some embodiments, the second funnel is advanced over the first funnel. In some embodiments, the method includes removing the second funnel from the compressed fenestrated graft. In some embodiments, removing the first funnel from the compressed fenestrated graft. In some embodiments, the first and second funnels are removed separately. In some embodiments, the fenestration template is at least partially planar, configured to be wrapped around the expanded graft. In some embodiments, the fenestration template is at least partially cylindrical and configured to at least partially receive the expanded graft.

In accordance with another aspect of the disclosure, a kit for modifying a graft (e.g. stent graft) is provided comprising: a template having at least one patient-specific feature and being sized and shaped complementarily with the graft; a ring; at least one suture configured to attach the ring to the graft; and a graft reloading device. In some embodiments, the at least one patient-specific feature is a hole. In some embodiments, the location of the at least one patient-specific feature is determined by the location of a pre-existing fenestration defined on the graft. In some embodiments, the template is cylindrical. In some embodiments, the template is at least partially planar, configured to be wrapped around the graft. In some embodiments, the template is printed on an insert that is packaged separately from the graft. In some embodiments, the ring is circularly shaped. In some embodiments, the size of the ring is adjustable. In some embodiments, the graft reloading device includes at least one funnel. In some embodiments, the graft reloading device includes at least one sleeve. In some embodiments, the at least one patient-specific feature is determined from a scan of a patient's anatomy. In some embodiments, the at least one patient-specific feature includes a hole complementary with a feature of the patient's anatomy. In some embodiments, the template is configured to receive an at least partially expanded graft. In some embodiments, the template imparts a marking of the at least one patient-specific feature location onto the graft. In some embodiments, the template is a plot of the at least one patient-specific feature location.

In accordance with another aspect of the disclosure, a method for reloading a graft that is partially expanded and partially deployed from a graft delivery device is profied comprising: advancing a compressing device to at least partially compress the partially deployed graft; and reloading the compressed graft into the graft delivery device. In some embodiments, the method includes forming at least one fenestration in the at least partially expanded graft. In some embodiments, forming the at least one fenestration includes causing the graft to be in an expanded configuration, and disposing a fenestration template over at least a portion of the expanded graft. In some embodiments, the template defines at least one patient-specific feature. In some embodiments, forming the at least one fenestration includes cauterizing a portion of the graft. In some embodiments, advancing the compressing device includes advancing a sleeve in a proximal direction over the partially deployed fenestrated graft. In some embodiments, the sleeve includes at least one handle disposed at a proximate end of the sleeve. In some embodiments, a portion of the sleeve is split upon deploying the graft into the expanded configuration. In some embodiments, the sleeve is advanced proximally to at least partially overlay the sheath of the delivery device. In some embodiments, the entire sleeve is advanced proximally to the graft, thereby deploying the graft into the expanded configuration. In some embodiments, the sleeve has an inner diameter larger than an inner diameter of a sheath of the delivery device. In some embodiments, the method includes coupling a fenestration ring to the fenestrated graft, the fenestration ring being complementarily sized and shaped with the at least one fenestration. In some embodiments, coupling the fenestration ring to the fenestrated graft includes at least one suture. In some embodiments, the patient-specific feature is determined from ascan of a patient's anatomy. In some embodiments, the at least one patient-specific feature includes a hole complementary with a feature of patient's anatomy. In some embodiments, the fenestration template is at least partially planar and configured to be wrapped around the expanded graft. In some embodiments, the fenestration template is at least partially cylindrical and configured to at least partially receive the expanded graft. In some embodiments, the sleeve has a plurality of handles disposed at a proximal portion and elongated cylindrical tube portion extending therefrom. In some embodiments, the sleeve is advanced to dispose the fenestrated graft within the elongated cylindrical tube portion of the sleeve. In some embodiments, the sleeve is formed of plastic.

In accordance with another aspect of the disclosure, a system for a reloading a medical device is provided comprising: an elongated shaft having a proximal end and a distal end defining a longitudinal axis therebetween, the elongated shaft including a sheath configured to retain a medical device therein; a handle; a rotatable knob coupled to the sheath, the rotatable knob configured to displace the sheath in a first direction when the rotatable knob rotates in a first direction, and to displace the sheath in a second direction when the rotatable knob rotates in a second direction; a reloading stop, the reloading stop having a first interface disposed on a proximal end and a second interface disposed on a distal end thereof; wherein the first interface of the reloading stop is configured to engage one of the rotatable knob and the elongated shaft, and the second interface of the reloading stop is configured to engage the other one of the rotatable knob and elongated shaft. In some embodiments, the first direction is clockwise, and the second direction is counterclockwise. In some embodiments, the first direction is counterclockwise, and the second direction is clockwise. In some embodiments, the reloading stop includes a channel disposed along a longitudinal axis thereof. In some embodiments, the channel extends from a proximal end to a location spaced from the distal end of the reloading stop. In some embodiments, the channel is configured to receive a threaded collar therein, with the threaded collar configured for displacement in the proximal and distal directions. In some embodiments, the reloading stop includes a rim disposed at a proximal end, the rim having a diameter larger than the reminder of the reloading stop. In some embodiments, the rim is disposed within the rotatable knob. In some embodiments, the reloading stop includes an internal protrusion disposed at a distal end, the internal protrusion extending normal to the interior surface of the reloading stop. In some embodiments, the reloading stop is coupled to the elongated shaft in a longitudinally-fixed position. In some embodiments, the reloading stop is removable from the elongated shaft. In some embodiments, rotation of the rotatable knob in the first direction displaces the sheath in a distal direction to expose the medical device, and rotation of the rotatable knob in the second direction displaces the sheath in a proximal direction to cover the medical device. In some embodiments, the fist direction is clockwise, and the second direction is counterclockwise. In some embodiments, the fist direction is counterclockwise, and the second direction is clockwise.

### BRIEF DESCRIPTION OF THE DRAWINGS

The accompanying drawings, which are incorporated into and constitute a part of this specification, illustrate various exemplary embodiments and together with the description, serve to explain the principles of the disclosed embodiments. The drawings are illustrative and are not necessarily drawn to scale, with some components and features being exaggerated for clarity. The drawings illustrate various aspects and features of the present subject matter and may illustrate one or more embodiment(s) or example(s) of the present subject matter in whole or in part.

FIG. 1 is a flow diagram illustrating a method for reloading a graft into a graft delivery system, in accordance with one or more embodiments of this disclosure.

FIGS. 2A-B are illustrations of exemplary fenestrated stent grafts, in a deployed and expanded configuration, in accordance with the present disclosure.

FIG. 3 is an illustration of an exemplary fenestrated stent graft in accordance with the present disclosure.

FIG. 4 is an illustration of an exemplary fenestration template in accordance with the present disclosure.

FIGS. 5A-H and FIG. 19 are illustrations of additional exemplary fenestration templates, including a way to package said template (see FIGS. 5G-H), in accordance with the present disclosure.

FIG. 6A is an illustration of an exemplary two-dimensional fenestration coordinate system, in accordance with the present disclosure.

FIG. 6B is an illustration of an exemplary fenestration ring attached to the graft via sutures, in accordance with the present disclosure.

FIGS. 7A-C are diagrams illustrating a method of imparting modifications to an exposed stent graft, in accordance with one or more embodiments of this disclosure.

FIG. 8A is an exemplary kit including funnels for reloading a stent graft device.

FIG. 8B is a process diagram illustrating the steps of reloading a modified graft, in accordance with one or more embodiments of this disclosure.

FIG. 9 is a schematic of a graft reloading kit, in accordance with one or more embodiments of this disclosure.

FIG. 10 is a schematic of a graft reloading kit, in accordance with one or more embodiments of this disclosure.

FIGS. 11-13 are diagrams illustrating another method of reloading a graft via a sleeve, in accordance with one or more embodiments of this disclosure.

FIGS. 14-17 are illustrations of an exemplary stent graft delivery device, including structure to facilitate reloading of the modified stent graft within the primary sheath of the device, in accordance with one or more embodiments of this disclosure.

FIG. 18 is an illustration of an exemplary stent graft deployment device.

### DETAILED DESCRIPTION

Reference will now be made in detail to the exemplary embodiments of the present disclosure, examples of which are illustrated in the accompanying drawings. Wherever possible, the same reference numbers will be used throughout the drawings to refer to the same or like parts.

The systems, devices, and methods disclosed herein are described in detail by way of examples and with reference to the figures. The examples discussed herein are examples only and are provided to assist in the explanation of the apparatuses, devices, systems, and methods described herein. None of the features or components shown in the drawings or discussed below should be taken as mandatory for any specific implementation of any of these devices, systems, or methods unless specifically designated as mandatory.

Also, for any methods described, regardless of whether the method is described in conjunction with a flow diagram, it should be understood that unless otherwise specified or required by context, any explicit or implicit ordering of steps performed in the execution of a method does not imply that those steps must be performed in the order presented but instead may be performed in a different order or in parallel.

As used herein, the term “exemplary” is used in the sense of “example,” rather than “ideal.” Moreover, the terms “a” and “an” herein do not denote a limitation of quantity, but rather denote the presence of one or more of the referenced items.

When reference is made herein to an aortic prosthesis, such as a “stent graft,” “prosthesis,” “stent graft prosthesis,” “vascular prosthesis” or other prostheses to be delivered or implanted in a patient, the word “proximal” means that portion of the prosthesis or component of the prosthesis that is relatively close to the heart of the patient, while “distal” means that portion of the prosthesis or component of the prosthesis that is relatively far from the heart of the patient.

When, however, reference is made to a delivery system or a component of a delivery system employed to deliver, or implant, a prosthesis, the word, “proximal,” as that word is employed herein, means closer to the clinician using the delivery system. When reference is made to a delivery system where a component of the delivery system is “distal,” as that term is employed herein, means further away from the clinician using the delivery system.

For clarity, the word “proximate” means “close to,” as opposed to the meanings ascribed to “proximal” or “distal” described above with respect to either the prosthesis or delivery system.

A stent graft is an implantable device made of a tube-shaped surgical graft covering and a balloon expandable or self-expanding frame, where the surgical covering is made from a textile (PET, ePTFE, Nylon etc.) and the frame is made from metals such as stainless steel, cobalt chromium, Nitinol etc.. In many designs, the stents are attached to textile using a surgical grade suture that can be made from multiple materials such as PET, UHMWPE, Nylon etc.. Finally, stent-grafts usually contain additional metallic elements intended to allow visualization under fluoroscopy (continuous x-ray). The stent graft is placed inside a blood vessel to bridge a weakened section of aortic wall or a partially or fully ruptured location of aortic wall, for example, an aneurismal, dissected, transected or other diseased segment of the blood vessel, and, thereby, either exclude the hemodynamic pressures of blood flow from the weakened segment of the blood vessel or prevent blood exiting the true lumen of the aorta through a partial or complete rupture of the blood vessel.

In selected patients, a stent graft advantageously eliminates the need to perform open thoracic or abdominal surgical procedures to treat diseases of the aorta and eliminates the need for total aortic reconstruction. Thus, the patient has less trauma and experiences a decrease in hospitalization and recovery times. The time needed to insert a stent graft is substantially less than the typical anesthesia time required for open aortic bypass surgical repair, for example.

Surgical and/or endovascular grafts have widespread use throughout the world in vascular surgery. There are many different kinds of vascular graft configurations. Some have supporting framework over their entirety, some have only two stents as a supporting framework, and others simply have the tube-shaped graft material with no additional supporting framework.

Current stent grafts are designed with a memory shape at the point of manufacturing (Examples of such “memory” shape materials are disclosed in U.S. Pat. No. 6,306,141 which is hereby incorporated by reference in its entirety). As such, these stent grafts are constantly trying to reform to the memory shape. Once the stent is exposed from the sheath during deployment, the stent expands to a larger shape than the delivery sheath. With the stent graft at its expanded diameter, the physician will be able to add the fenestrations using the template tooling. However, with the stent graft at its expanded diameter, it now becomes a challenge to place the stent graft back into its original delivery sheath. Conventionally, physicians will use a time intensive system of manually wrapping the stent graft down using surgical tapes, wires or other similar materials and then slowly replacing the stent graft into its original delivery sheath while slowly unwinding the tape/wire/other material from the stent graft at the same time. To address this shortcoming, a funneling system is introduced along with a set of specifically designed tubes. The introduction of multiple tubes designed to step down the stent graft diameter uniformly re-collapse the exposed stent graft. The reshaping of the stent graft structure brings the structure's diameter back to a diameter able to be placed back into the primary sheath. The use of the funnel and tube system allows for a process that has significantly more control that the conventional process described above. The controls of this process include the actual diameter that the stent graft is re-compressed to as well as the materials that come into contact with the stent graft through the process. Especially in cases where physicians use surgical wires to wrap down the stent graft, the chance for damage to any of the main components of the stent graft (graft material, stents or suture) is high, and could present a risk to the long term durability of the implanted device. The preferred materials for the tooling described significantly mitigate the risk associated with damaging the device.

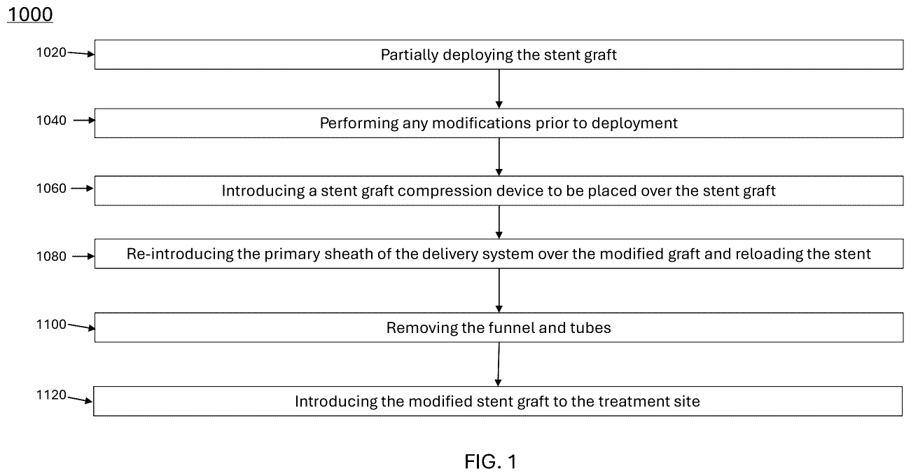

FIG. 1 is a flow diagram 1000 illustrating a method (i.e., steps 1002-1120) for reloading a graft into a graft delivery system. In step 1020, the method includes at least partially deploying the stent graft. “Partial deployment” refers to a user removing the stent from a restrictive packaging and allowing for the stent graft to diametrically expand out of the primary sheath, as it would during a procedure prior to placement within the patient. The stent graft is thereby partially exposed, which allows for the user to perform any necessary modifications to the partially exposed stent prior to final deployment (as performed in step 1040). In some embodiments, the stent graft can be fully expanded (e.g. the restrictive sheath of the deployment device being fully retracted in the proximal direction to expose the entire stent graft) so that the desired modifications can be performed/incorporated at any location along the stent graft. Reference herein to a “partially” exposed stent graft is to be understood as a non-limiting recitation, and can include a “fully”exposed stent graft.

In step 1040, the user performs any modifications necessary on the partially exposed stent. For example, such modifications can include adding fenestrations to a standard (e.g. previously assembled, or “generic”) stent graft, as described in further detail below. Other modifications or enhancements that could be made to a stent graft include: Shortening of the length of the stent graft, removal of a component-e.g. a bare stent-from the stent graft, addition of branches to a stent graft, administration of a coating to introduce a biologically active agent to the stent graft to induce a tissue response, administration of a coating to introduce an anti-coagulative active agent to the stent graft to prevent thrombus or clotting, or administration of a coating to introduce a antibiotic active agent to the stent graft to prevent infection. On competitive devices if the fenestration falls over a stent strut the physician will physical move the strut out of the way and sew it into a new location that does not impede the fenestration location.

In step 1060, the method can include introducing a stent graft compression device (e.g. funnel(s) or sleeve) to be placed over the stent graft. The compression device re-constrains the stent graft to a diameter that will allow for the primary sheath of the deployment device to be advanced over the, now modified, stent graft so that it can then be deployed within a patient. In some embodiments, a secondary compression device (e.g. funnel or tube of a smaller diameter) can be employed to be placed over the constrained graft, so that the reduction in stent graft diameter is done in stages, thereby minimizing the stress on the stent struts via an iterative compression process.

In step 1080, the method can include reintroducing the primary sheath of the delivery system over the modified graft and reloading the stent. The primary sheath may be reintroduced by pulling the primary sheath back over the modified graft before reloading the stent.

In step 1100, the method can include removing the compression device(s) and any tubes from the stent graft device.

In step 1120, the method can include introducing the modified stent graft to the treatment site for deployment.

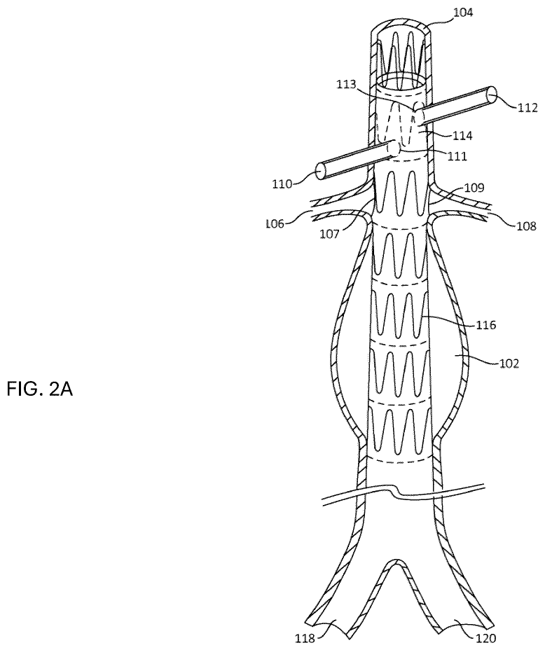

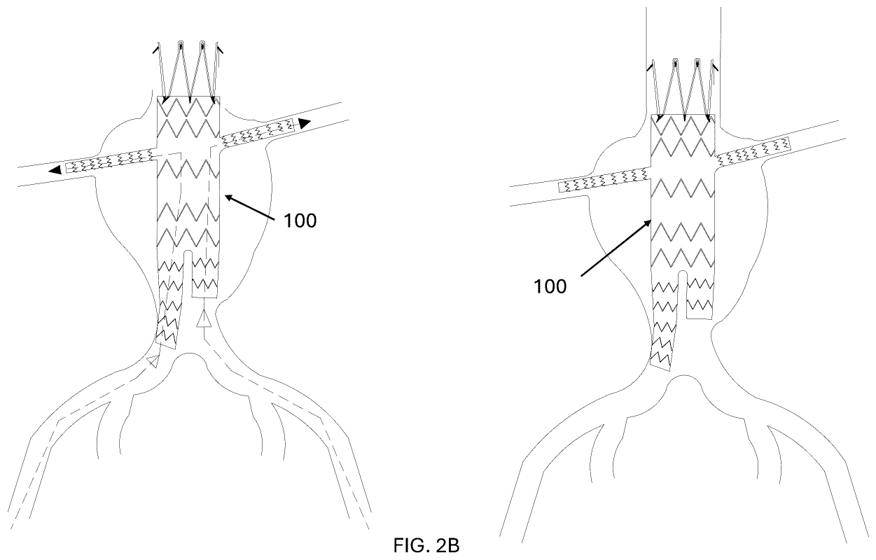

FIGS. 2A-B illustrates exemplary stent grafts 100 deployed in an abdominal aorta (though the present disclosure includes stent grafts for deployment in additional/alternative arteries), in accordance with at least one embodiment. As shown, the stent graft is deployed inside an aorta 104 to treat an aneurysm 102 that extends from below the renal arteries 106 and 108. The stent graft typically has a tubular body and comprises a plurality of internal 114 and/or external 116 stents or stent-like structures (collectively referred to as stent struts) supporting a graft material that is typically biocompatible. In some cases, the stent graft may have bifurcated legs (see FIG. 2B) extending into one or more additional branch vessels such as iliac arteries 118 and 120.

### Fenestrations and Template

In some embodiments, the stent graft provides fenestrations (boles) to allow blood flow through the stent graft into side branch vessels. For example, in cases involving juxarenal aneurysms, the non-dilated portion of the aorta proximal to the aneurysm is typically too short to provide a reliable seal between the stent graft and the aorta. In such cases, the proximal end of the stent graft needs to be placed higher in the non-dilated portion of the aorta. Thus, as shown in FIG. 2, the proximal portion of the stent graft provides fenestrations such as 107 and 109 to allow flow through the stent graft into the renal arteries 106 and 108, respectively. In some embodiments, the locations of the fenestrations are selected to avoid overlapping with the stent struts. Depending on the deployment position of the stent graft, in some cases, the stent graft may also include additional fenestrations such as 111 and 113 to accommodate other branch vessels such as superior mesenteric artery (SMA) 110 and celiac artery 112.

Generally, a precise fit between the fenestrations of the stent graft and the openings of the branch vessels is important both for ensuring the flow between the aorta and the branch vessels and for excluding the flow to the aneurysm. To ensure a precise fit, a stent graft for a particular patient is preferably fenestrated accordingly to that patient's particular anatomy.

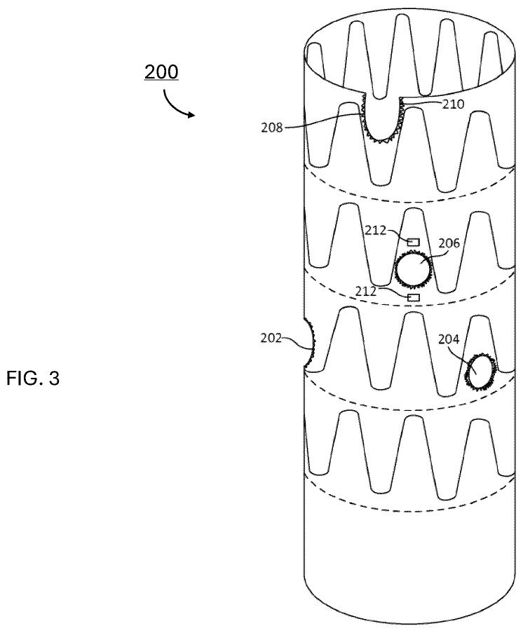

FIG. 3 illustrates an example fenestrated portion 200 of a stent graft, in accordance with at least one embodiment. As discussed above, a stent graft may include one or more fenestrations configured to accommodate one or more branch vessels when the stent graft is deployed in an aorta. A fenestrated portion of the stent graft includes at least one fenestration. The fenestrated portion may be located near a proximal end, a distal end or any portion of the stent graft where branch vessels need to be accommodated. In this example, the fenestrated portion of the stent graft includes two fenestrations 202 and 204, each corresponding to a renal artery. The fenestrated portion may include additional fenestrations for other branch vessels that may be otherwise blocked by the unfenestrated stent graft. For example. the fenestrated portion 200 includes fenestrations 206 and 208 (which can be circular, “scalloped”or U-shaped, and/or any other desired geometry) to accommodate the superior mesenteric artery (SMA) and celiac artery, respectively. In other embodiments, a stent graft may include fenestrations to accommodate more or fewer branch vessels than illustrated here. For example, a stent graft may include fenestrations to accommodate the inferior mesenteric artery (IMA), internal iliac arteries, and the like.

In various embodiments, the fenestrations in a stent graft may be of any suitable sizes or shapes. In typical embodiments, the fenestrations are sized and/or shaped to accommodate the corresponding branch vessel openings. For example, the fenestrations may be substantially circular if the corresponding branch vessel is otherwise covered entirely by the graft material of the deployed stent graft. Such may be the case when the branch vessels are located away from the ends of the stent grafts. For example, as shown, fenestrations 206 and 208 for the renal arteries are substantially circular. On the other hand, the fenestration may be partially circular if the corresponding branch vessel is only partially covered by the graft material during deployment. Such may be the case when the branch vessels are located near an end of the stent grafts. For example, as shown, fenestration 202 for the celiac artery is only partially circular or U-shaped to accommodate only a portion of branch vessel opening that is blocked by the graft material. In yet other embodiments, the fenestrations may have non-circular shapes.

In various embodiments, the peripherals or edges of the fenestrations may be reinforced wholly or partially to provide stability, for example, for anchoring of stent grafts into the branch vessels. In an embodiment, the peripheral of a fenestration may be stitched or sutured using wires. In another embodiment, the peripheral of a fenestration may be coupled (e.g., via stitches) to a ring or a similar support frame.

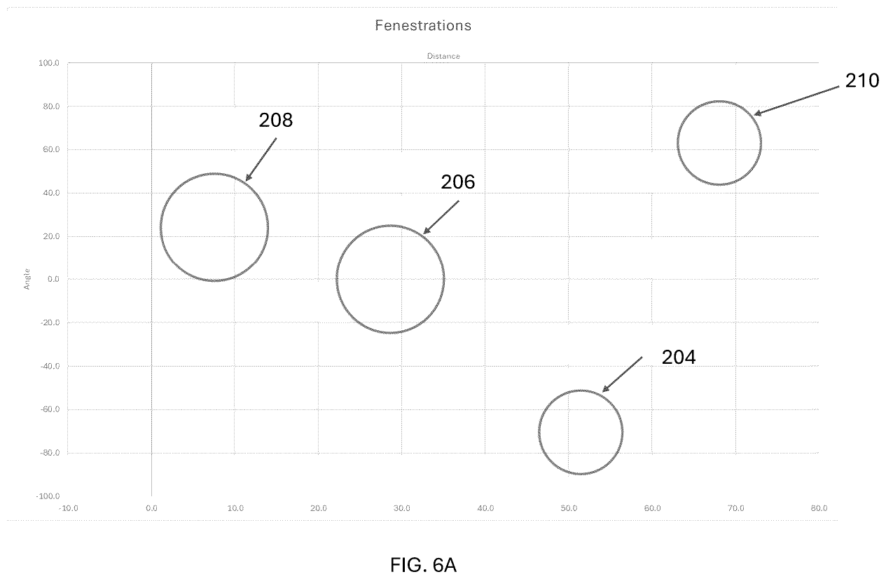

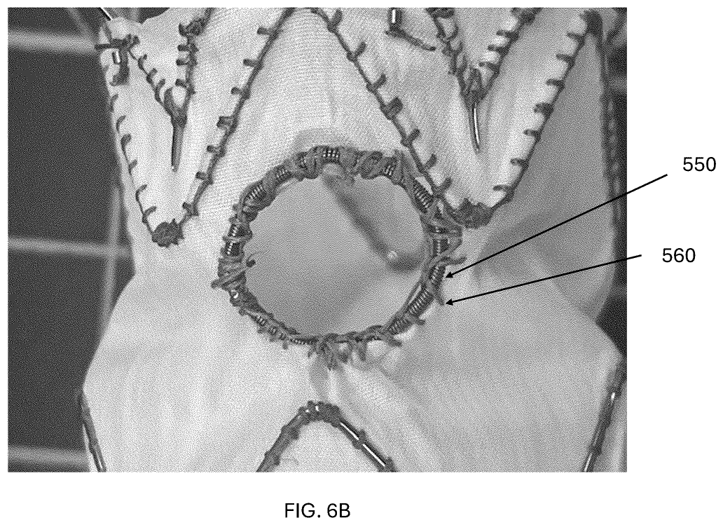

In various embodiments, the boundary of the fenestrations may be marked to facilitate visual tracking of the fenestrations during (final, or after the initial deployment of the graft wherein the fenestrations are formed in the graft) deployment of the stent graft. For example, the peripheral of the fenestrations may be sutured using gold wires 210 or wires of other radio-opaque materials. Similarly, the location of the fenestration may be marked by one or more radio-opaque markers 212. Alternatively, radiopaque fenestration rings can be coupled to the graft to surround the fenestration and provide visibility to the physician, as described in further detail in connection with FIG. 6B. Such radio-opaque wires or markers may facilitate fluoroscopic visualization of the fenestrations during the repair procedure and allow a physician to locate the fenestration with respect to the corresponding branch vessel.

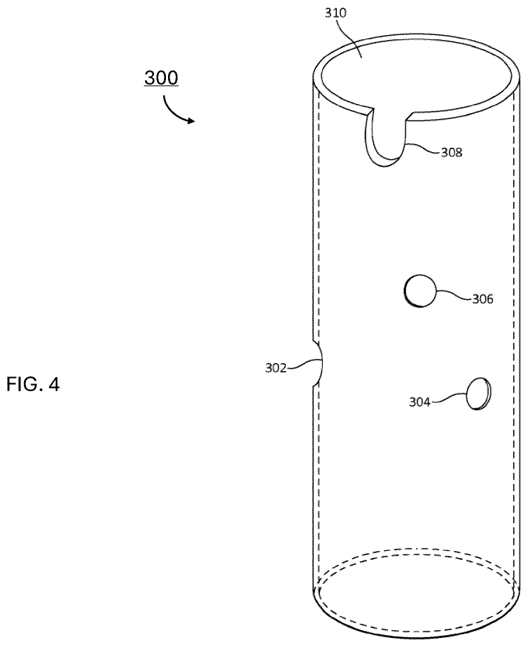

FIG. 4 illustrates an example fenestration template 300 that may be used to generate fenestrations on a stent graft, in accordance with at least one embodiment. For example, the fenestration template 300 may be used to generate fenestrations illustrated in FIG. 3. The fenestration template typically includes one or more branch openings corresponding to the openings of one or more branch vessels in an aorta. As illustrated, the fenestration template 300 includes branch openings 302 and 304 for the renal arteries as well as openings 306 and 308 for the superior mesenteric artery (SMA), and celiac artery, respectively. The diameters of the branch openings on the fenestration template may correspond to the diameters of actual openings of branch vessels in a patient's aorta, or may be of a predefined value (e.g., 4 mm ˜10mm). In other embodiments, the fenestration template may include more or fewer branch openings than illustrated in the exemplary embodiments shown.

Generally, the shape of the fenestration template corresponds to the lumen of an aorta segment that encompasses one or more branch vessels. Hence, the fenestration templates typically have a tubular or cylindrical shape. In some embodiments, such as illustrated in FIG. 4, the fenestration template may include a lumen 310 that corresponds to the lumen of an aorta. The diameter of the lumen 310 may be determined based on a diameter of the stent graft to be used with the fenestration template or a predefined value, corresponding to the oversizing requirements of the stent graft being modified. When in use, in some embodiments, the fenestration template may be slid over a stent graft such that the stent graft is at least partially inside the lumen of the fenestration template.

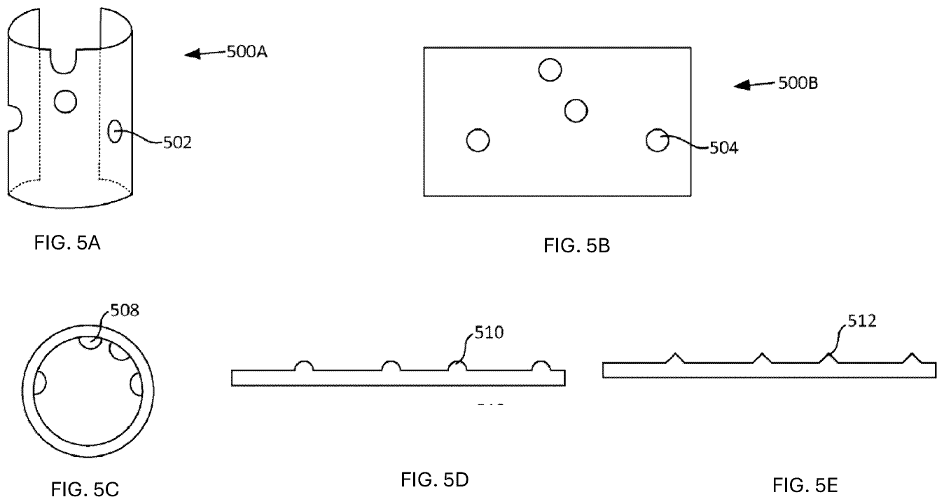

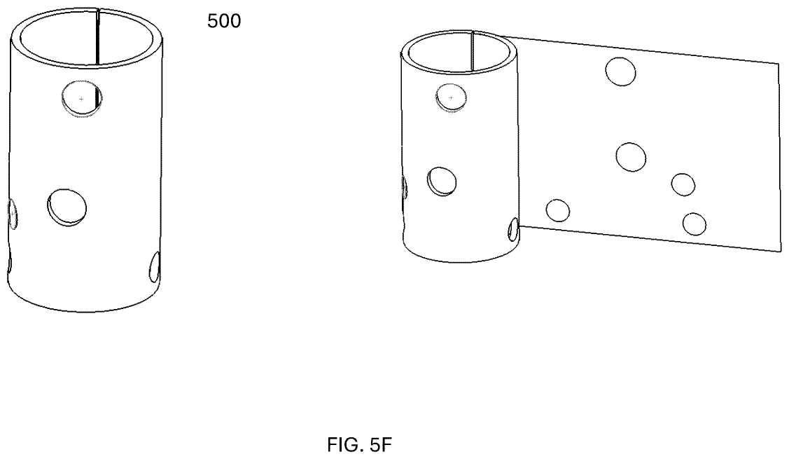

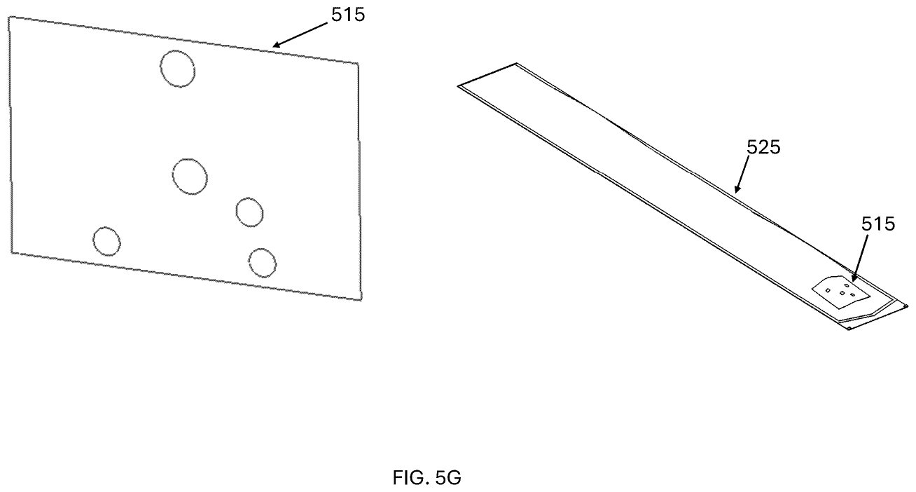

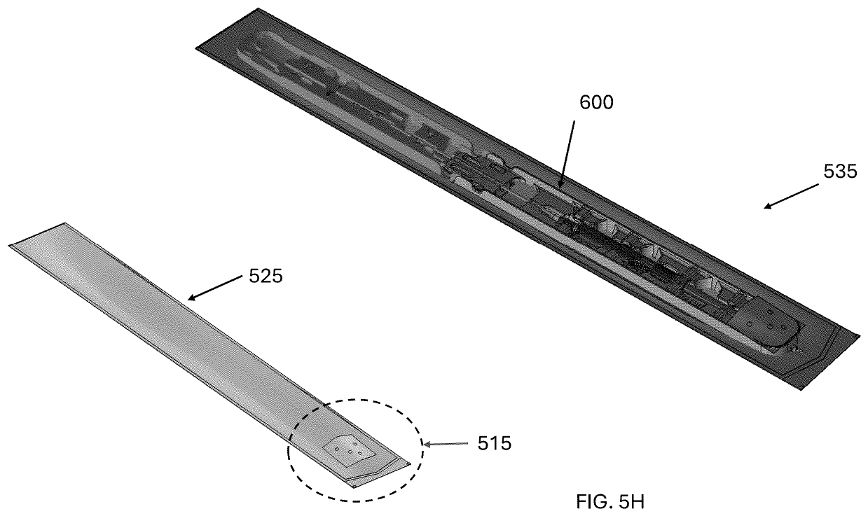

FIGS. 5A-H illustrate more examples of fenestration templates, in accordance with some embodiments. For example, as shown in FIG. 5A, instead of a tubular shape, the fenestration template 500A can have a partial tubular shape (e.g., semi-circular) with an opening along a longitudinal axis. In some embodiments, the fenestration template may be made of an elastic or resilient material such that the fenestration template may open along the longitudinal axis and clasp around a portion of a stent graft. In other embodiments, the fenestration template may be made of a rigid material and the fenestration template may slide over a stent graft in a similar fashion as discussed in connection with FIG. 4.

As shown in FIG. 5B, in some embodiments, the fenestration template 500B can be a flat sheet with holes 504 corresponding to the openings of branch vessels on an aorta. In some embodiments, the fenestration template may be made of any elastic material such as paper, metal foil, plastic film, and the like. In some embodiments, the flat sheet may be made of biocompatible graft material. When in use, the fenestration template may be wrapped around a portion of a stent graft that needs to be fenestrated such that the holes 504 on the fenestration template may be used to produce (e.g. pierce the graft material) the required fenestrations. In some embodiments, where biocompatible graft material is used as fenestration template, such that the fenestration template may be wrapped around uncovered bare metal stents to produce a fenestrated stent graft. In some embodiments, the fenestration template may include one or more markings (e.g., circles) designating the branch vessels instead of or in addition to the holes.

In some other embodiments, such as shown in FIG. SC, the fenestration template 500C may include one or more protruding structures on an inner surface of the template. When in use, in an embodiment, the fenestration template 500C may be placed outside a stent graft such that the template overlaps with a portion of the stent graft to be fenestrated.

As shown in FIG. SD, in some embodiments, the fenestration template 500D may be similar to fenestration template 500B discussed in connection with FIG. 5B except that the holes are replaced by protruding structures such as discussed in connection with FIG. 5C. When in use, in an embodiment, the fenestration template 500D may be wrapped around a portion of a stent graft to be fenestrated such that the protruding structures point against the stent graft. These protruding structures can impart a marking (e.g. dent) in the graft to signal where the fenestration is to be formed (e.g. via cauterization once the template is removed) and/or pierce the graft material so as to form the fenestration with the template still installed on the expanded stent graft).

In various embodiments, the protruding structures discussed above may be of any suitable dimensions and/or shapes. For example, the protruding structure may have a semi-circular shape as shown in FIGS. 5C-D. For another example, the protruding structure may have a pointed tip as shown in FIG. 5E.

As noted above, in some embodiments, the protruding structures may be configured to mark locations of the fenestrations on the stent graft material upon contact. Alternatively. or additionally, the protruding structures may be configured to produce fenestrations on the stent graft material, for example, using thermal, mechanical, chemical, or other means. For example, the protruding structures may be heated (e.g., electrically) to act as thermal cautery tools for generating holes in the graft material. For another example, the protruding structures may have sharp tips usable for puncturing apertures in the graft materials.

In various embodiments, the fenestration templates may be made of one or more suitable materials, rigid or non-rigid, such as thermoplastic, plaster, metal alloy, titanium alloy, paper, metal foil, plastic film, photopolymer, and the like. In some embodiments, a smooth coating material may be applied to a surface (e.g., lumen wall or outer surface) of the fenestration template to facilitate easier interfacing with a stent graft.

In various embodiments, various aspects of the fenestration templates such as the dimensions of the holes, openings, protruding structures, and the like, the dimension of the template and the like may be determined based on the dimensions of the actual aorta or branch vessels or configurable (predefined) values.

In various embodiments, the fenestration templates may be manufactured using any suitable technologies such as 3-D printing or additive prototyping/manufacturing technologies, subtractive manufacturing techniques, 2-D printing, and the like or a combination thereof. In some embodiments, the fenestration templates are generated for patient-specific anatomy, for example, based on patient-specific imaging data as described in U.S. Patent No. 9,811,613—the entirety of which is hereby incorporated by reference. For example, and as shown in FIG. 6A, in some embodiments a template can be constructed from a 2-D rendering of a patient's anatomy with an origin (point “0.0”) of the template fixed or mapped to that patient's superior mesenteric artery (SMA), which is a major artery of the abdomen, and the coordinates (e.g. X, Y, Z dimensions and relative angles in a cartesian coordinate system) can be provided to the fenestration location(s), and/or other anatomical structures of the patient.

In the exemplary embodiment of the template shown in FIGS. 5F-H the patient-specific fenestration locations are marked on the template 515 with indica only (e.g. ink or removable sticker), without removing any material from the template initially. The fenestration indicia/markings can be imparted to the template while it is in a generally flat configuration, as this ensures accuracy of the fenestration size/location. In such embodiments, 3-dimensional scans of the patient's anatomy are converted into a 2-dimensional representation, with the fenestration location/geometry mapped to the 2-dimensional model. After providing the template with the patient-specific fenestrations, the flat template can then be converted or rolled into a tubular shape to serve as a sleeve, which can be advanced over the (non-fenestrated) stent graft.

The patient-specific template can then be packaged, e.g. in a sterile packaging 525, as shown in FIG. 5G. and shipped to the local physician who can: i) create the fenestrations holes within the template (though in some embodiments the holes can be pre-formed in the template), ii) expose the non-fenestrated stent graft, iii) place the template over the stent graft, and iv) form fenestrations within the graft, and v) re-insert the now fenestrated graft within the sheath of the deployment device (as described in further detail below). This allows for a generic, non-fenestrated, stent graft to be shipped along with the patient-specific fenestration template (having holes pre-formed on the template, or just hole markings/indicia printed thereon) to expedite delivery of the device to the physician/patient in need. The customization of the generic stent graft can be performed on site, significantly reducing the lead time required to get patient-specific stent grafts to patients in need. As shown in FIG. 5H, the patient-specific template 515 can be contained in a first package 525, which can be included in the package 535 including the generic non-fenestrated stent graft delivery device 600.

### Fenestration Ring and Sutures

In some embodiments, a fenestration can be created in a stent graft using any suitable method. For example, the graft material within the boundary of the fenestration edge/circumference can be cauterized. Cauterization can be advantageous in that it prevents unravelling of the stent graft fabric at the edge(s) of the fenestration. Additionally, or alternatively, a number of cuts can be created to segment the graft into the desired number, and shape, of fenestrations. In some embodiments the material cut from the graft is removed/discarded. In some embodiments, the material cut from the graft can remain partially attached, e.g. a flap, and folded back on the exterior surface of the graft.

Additionally, a ring can be attached to the graft which circumscribes, or otherwise surrounds, the fenestration. An exemplary embodiment of a ring 550 surrounding the fenestration is shown in FIG. 6B. In some embodiments the ring is attached to the graft after the fenestrations are formed. In other embodiments the ring is attached before the fenestrations are formed (but after the location of the fenestrations are identified on the graft). As noted above, the fenestration ring can be formed of a radiopaque material to provide visibility of the location, and relative dimensions, of the fenestration. The ring can be provided as a closed loop, or alternatively an elongate string-like element that can be formed/shaped into a loop when attaching to the graft. In some embodiments the rings are elastic and/or deformable to adjust the size and shape to mimic the contours of the graft. Alternatively, the rings can be rigid to resist local deformations and retain a generally uniform shape. The fenestration rings can be provided in a variety of sizes, e.g. diameters ranging from 4 mm˜12 mm; and/or of set sizes, such as 6 mm and 8 mm.

One or more suture threads 560 can be used to attach the fenestration ring to the graft. The suture thread can also include a material with radiopaque properties, for visualization of the suture thread using radiographic imaging. The sutures can be made of synthetic filaments (single, poly, braided, etc.) and/or metallic thread. Alternatively, or in addition to the suture thread, fasteners such as, for example, staples, rivets, micro-rivets. adhesives, and/or welding can be used to secure the fenestration ring to the graft. Likewise, the fasteners can include or be formed of material with radiopaque properties such that the fasteners are visible using radiographic imaging.

In accordance with an aspect of this disclosure, a “kit” can be provided for use in forming patient-specific fenestration(s) to a universal stent graft (i.e. no fenestrations initially included), with the fenestration kit including:
i) a patient-specific template as described above (which can be removably coupled to the stent graft once exposed from the sheath);
ii) at least one fenestration ring (which can be attached to the fenestration edge/boundary)
iii) at least one suture (which can be a closed loop, or open thread and used to attach the fenestration ring to the stent graft)

The provision of a kit provides multiple benefits to the physician and patient. The kit enables the physician to have all of the tools necessary to partially deploy, position the fenestrations in the proper place and properly reload the system in a consistent and efficient manner. More importantly, however, the provision of a kit ensures that the materials used to modify the device, especially the suture, have been tested for both acute and long term integrity. The act of reloading a device can cause significant stresses on both a newly introduced suture and the newly introduced fenestration ring. Additionally, the long-term fatigue of the system (including the integrity of the suture, the fenestration ring as well as the bridging stent when inserted into the fenestration ring) is of concern. By using pre-manufactured-supplier provided materials, the physician has the confidence that the materials of construction have been tested for all reasonably anticipated use conditions, and the short and long term integrity of the system should be appropriate for clinical use. As there have been known systemic failures attributable to inappropriate suture design / choice, thus the proper testing of such a system, which the present disclosure provides, is critical to the long term success of the procedure.

In some embodiments, this kit can be designed for single use.

### Forming Fenestrations in Assembled Stent Graft

In accordance with an aspect of the disclosure, fenestrations can be incorporated into a previously assembled stent graft, without disassembly of the device. Additionally, fenestration rings can be attached to the graft, e.g. via sutures as described above, to circumscribe or otherwise surround the fenestration(s), without disassembly of the device.

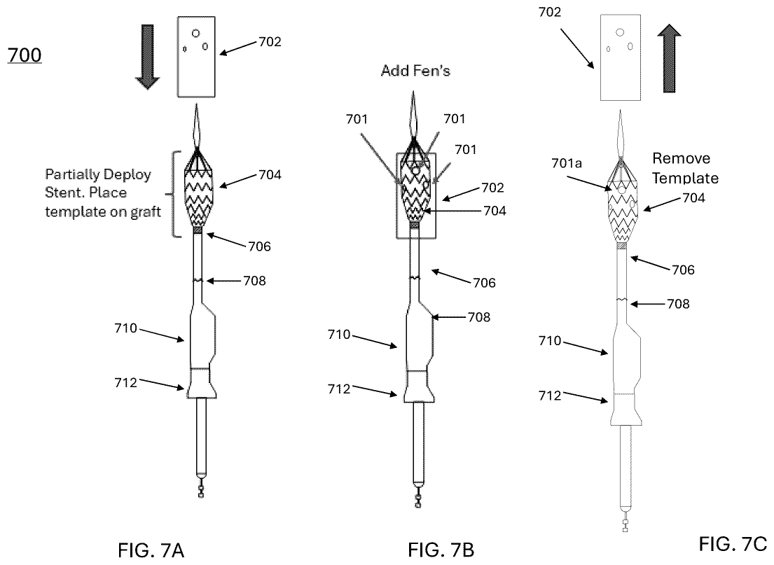

FIG. 7A-C are diagrams illustrating an exemplary method of placing a template on an exemplary stent graft device 700. There are various features characterizing a stent graft. The first significant feature is the tube of graft material. This tube is commonly referred to as the graft and forms the tubular shape that will, ultimately, take the place the diseased portion of the blood vessel. As described above, the graft can be made of a woven sheet (tube) of polyester or PTFE. The circumference of the graft tube is, typically, at least as large as the diameter and/or circumference of the vessel into which the graft will be inserted so that there is no possibility of blood flowing around the graft (also referred to as endoleak) to either displace the graft or to reapply hemodynamic pressure against the diseased portion of the blood vessel. Accordingly, to so hold the graft, self-expanding frameworks are attached typically to the graft material, either on the interior or exterior thereof. In some embodiments, the framework(s) located at the critical sealing junctions for anatomical branches, fenestrations, etc. can be disposed (e.g. sewed) to the interior surface of the graft, while the framework(s) located at less critical locations on the graft are disposed (e.g. sewed) to the exterior surface of the graft. Such a configuration maximizes the graft performance (e.g. sealability, flow through lumen, rigidity, etc.) while simplifying manufacture (since the majority of framework(s) can be disposed on the more easily accessible outer surface of the graft). The ridges formed by such an exterior framework help to provide a better fit in the vessel by providing a sufficiently uneven outer surface that naturally grips the vessel where it contacts the vessel wall and also provides areas around which the vessel wall can endothelialize to further secure the stent graft in place.

FIG. 7A illustrates a step of placing a template 702 on a partially (or completely) deployed stent graft. Thus, in accordance with an aspect of the disclosure, fenestrations can be added to a stent graft that is only partially expanded, or alternatively to a fully expended stent graft, if so desired. FIG. 7B illustrates a step of adding fenestration locations to the stent graft 704, as described in step 1040 of process 1000. In some embodiments the template is only used to mark the locations of the to-be-formed fenestrations; with the template removed before the fenestrations are formed in the stent graft. In some embodiments the fenestrations are formed with the template still positioned over the stent graft, with the template removed after the fenestrations are formed. FIG. 7C illustrates the removal of the template 702 from the fenestrated stent graft 704.

In FIG. 7A, stent graft deployment device 700 comprises a stent graft 704, a sheath tip 706, a primary sheath 708, a distal grip 710, and a turning knob 712. The stent graft 704 can comprise any standard use stent graft, including but not limited to a TREO® device manufactured by Terumo, Inc. The typical stent graft, for example stent graft 704, has a bifurcated tubular body and a circumferential framework including a series of rings along the tubular graft. In the preferred embodiment using the TREO system, the distal ends of each gate of the bifurcated portion of the stent graft will remain constrained in the delivery sheath to facilitate reloading.

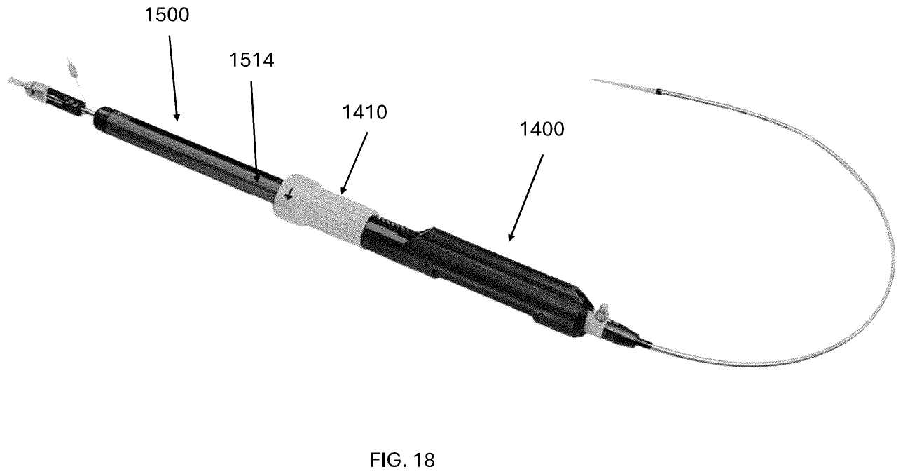

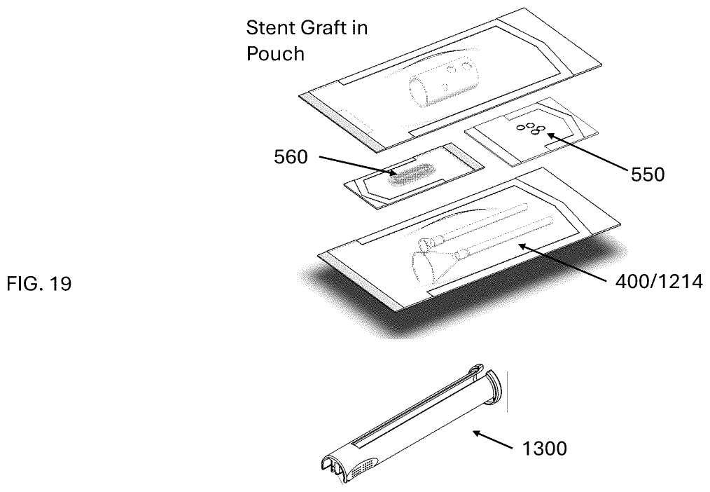

The tubular sheath tip 706 shares a central axis with the stent graft 704, and is located at the distal-most end of the sheath, and may provide a connection point with a proximal end of the stent graft 704. The diameter of the sheath tip 706 is smaller than the diameter of the stent graft 704 (when in the expanded configuration shown). The primary sheath 708 extends proximally from the sheath tip and is likewise tubular, sharing a similar or same diameter with the sheath tip 706. The distal grip 710 allows the user to grip the stent graft device 700 comfortably to manipulate the instrument as needed for deployment of the stent graft within the patient. The distal grip 710 has a larger radius to clearly indicate where the user is to hold the device. The turning knob 712 is used to adjust the device 700 (e.g. location of stent graft, articulation of guidewire, and/or displacement of sheaths) as needed, and can attach or detach downstream additions from the device. An exemplary deployment device for stent grafts is disclosed in U.S. Pat. No. 11,382,779, the entire contents of which are hereby incorporated by reference. In accordance with an aspect of this disclosure, the deployment device is able to reload the stent graft by the interaction of the turn knob 712 with the lead screw and handle grip (1400 in FIG. 18). Having the ability to move the turn knob 712 (1410 in FIG. 18) independently from the grip 1400 as well as over the lead screw in a way that does not actuate the sheath moving relative to the graft. As shown in FIGS. 7A-C, the stent graft 704 is partially deployed, as indicated by the stent graft's radial expansion.

In FIG. 7B, the template 702 is slid onto the device 700 to at least partially cover the stent graft 704. The template 702, which features a series of fenestrations 701, can be used to provide guidance to the user for marking on or making holes in the stent graft 704. In some embodiments the stent graft is entirely expanded (e.g. sheaths are fully retracted) before the fenestration template 702 is coupled to or slid over the stent graft. In other embodiments the stent graft 704 can be only partially expanded (e.g. sheath still overlapping a portion of the stent graft) when the fenestration template 702 is coupled to or slid over the stent graft with the amount of overlap between template and graft being sufficient enough to position the template holes at the desired location of the stent graft to thereafter form the fenestration.

In some embodiments, all fenestrations are formed simultaneously. In other embodiments each fenestration is formed separately, and each fenestration can be formed in sequence (e.g. from the distal end towards the proximal end of the stent graft), with the template being advanced over the stent graft in an indexed or stepped fashion from a first fenestration location to a second (proximal) location. For example, the template 702 can be slid further in a proximal direction along the graft 704 after each fenestration is made.

In FIG. 7C, after the desired fenestration locations are at least marked (and in some instances the fabric of the stent graft removed to actually form the fenestration) the template is displaced distally for removal from the stent graft 704. As shown in FIG. 7C, a user can fenestrate stent graft 704, where the fenestrations 701a correspond to the fenestrations 701 on the template 702. Fenestration ring(s) can be coupled to the stent graft (e.g. via sutures as described above), and the stent graft 704 can then be re-sheathed and ready for deployment into the patient.

### Re-Insertion of Modified Stent Graft Into Deployment Device With Funnel(s) as Compression Device

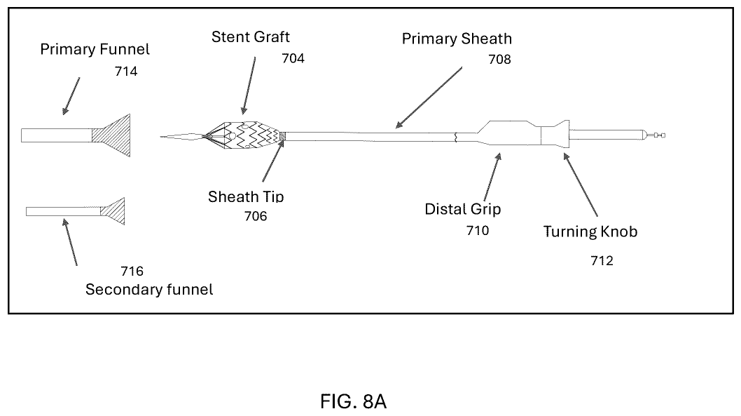

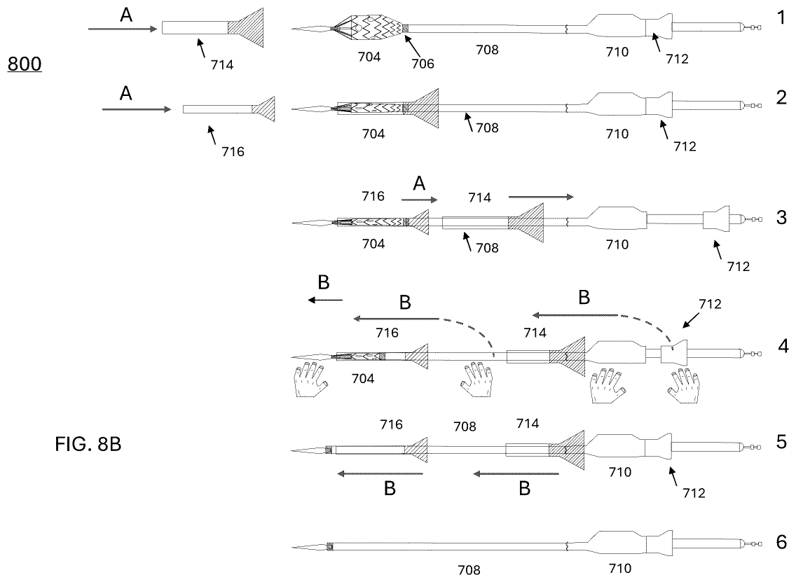

FIG. 8A is an exemplary “kit” for reloading a stent graft device (which can be designed for single use, e.g. the funnels are consumable/disposable items). The kit can be used with a stent graft 704, a sheath tip 706, a primary sheath 708, a distal grip 710, and a turning knob 712, as shown in FIGS. 7A-C, as well as primary funnel 714 and secondary funnel 716. The primary funnel 714 can have a larger diameter (e.g. frustoconical proximal portions and elongated cylindrical tube portions, respectively) than the secondary funnel 716. The differing size diameters of the funnels serve to gradually (e.g. two-step process) to compress the now fenestrated stent graft 704 back to its collapsed condition for reinsertion into the sheath(s), as described in further detail below. For purpose of illustration but not limitation, some exemplary size primary and secondary funnels include 19 Fr and 18 Fr, respectively. Each funnel has an enlarged first end (e.g. frustoconical proximal portion) and a smaller second end (e.g. elongated cylindrical tube portion) with a tapered section therebetween. Although not shown in FIG. 8A, the kit can also include fenestration rings and sutures.

Although the exemplary embodiments illustrated herein depict the modification of the stent graft to include the formation of fenestrations, additional or alternative modifications (application of radiopaque markings, drug eluting coatings, etc.) can be performed on the stent graft, That is, the present disclosure provides a universal system and method for re-inserting a stent graft that has been previously expanded and modified in some way, with the scope not limited to just formation of fenestrations.

FIG. 8B is a process diagram 800 illustrating the steps of reloading a modified (e.g. fenestrated) graft, for example the stent graft device 700 with the kit. As shown, the sheath 708 has been retracted (i.e. moved proximally) to uncover the stent graft 704 (which results in expansion of the stent graft). After any desired modifications (e.g. fenestrations) are performed on the stent graft, Step 1 of the re-insertion process commences, as shown in FIG. 8B, comprises placing the primary funnel 714 over the modified (e.g. fenestrated), at least partially exposed and expanded stent graft 704. The primary funnel 714 may be advanced in direction indicated by arrow “A” in step 1 of FIG. 8B by pushing (e.g. manually by hand) the primary funnel 714 over the stent graft proximally, wherein the diameter of the primary funnel 714, at its first end or “mouth”, is larger than that of the stent graft 704. In some embodiments, a lubricant (e.g. water, saline, oil, etc.) can be applied to the outer surface of the modified stent 704 to reduce friction between the stent graft and the internal surface of the primary funnel 714. Alternatively, in some embodiments the sheaths have a hydrophobic coating on the inner diameter to reduce friction and facilitate reloading.

As the primary funnel 714 advances over the stent graft 704, the taper of the primary funnel compresses or reduces the size of the stent graft from its fully expanded configuration with maximum size/diameter to an intermediate configuration with intermediate size/diameter that coincides with the second end of the primary funnel diameter. In the intermediate configuration, the modified stent graft still has a larger diameter than the sheath(s) so the modified stent graft cannot, yet, be reinserted into the deployment device.

The primary funnel 714 can be advanced a distance such that entire modified stent graft 704 passes through the tapered section of the primary funnel 714, with the modified stent graft residing within the narrower constant diameter section of the primary funnel 714. In use, the system traverses a 0.037″ diameter wire to navigate to the treatment site. For reloading, the wire adds support to prevent kinking of the system if excessive force was encountered when compacting the stent graft down into the funnel/Teflon tubes.

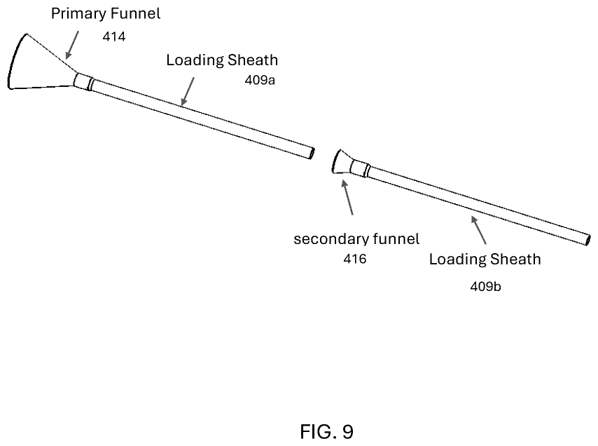

Step 2 of the process 800 comprises placing a secondary (smaller diameter) funnel 716 against the primary funnel 714. In some embodiments, the thereby primary funnel 714 can be moved further proximally, as shown by arrow “A” in FIG. 8B such that the two tapered portions of the respective funnels do not abut or engage each other. In such embodiments, the smaller secondary funnel 716 is advanced over the tubular (i.e. smallest and constant diameter) portion of the primary funnel 714 (also referred to as a loading sheath 409a in FIG. 9). As the secondary funnel 716 advances, this further compresses the graft 704, from the intermediate state (as compressed by the primary funnel 714) to the completely collapsed configuration wherein the modified stent graft 704 has its smallest diameter which is smaller than the diameter of the sheath(s), thereby permitting the modified stent graft 704 to be re-inserted into the deployment device. The operator can visually determine when the funnel(s) have been advanced a sufficient distance. In some embodiments, the funnels can include indicia (e.g. varied colors, graduated markings of distance, etc. on their tubular portions, or loading sheath 409a,b as shown in FIG. 9) to convey to the operator the degree of insertion. In some embodiments, the secondary funnel 716 is advanced to be concentric with the primary funnel 714 (i.e. both funnels are located at same location along the deployment device). In some embodiments, the secondary funnel 716 is only advanced over the tubular portion of the primary funnel 714 (or loading sheath 409a as shown in FIG. 9).

In some embodiments, once the secondary funnel 716 is advanced over the modified stent graft 704, the primary funnel 714 can be removed (i.e. advanced proximally) so that the end of the tubular (i.e. smallest and constant diameter) portion of primary funnel 714 is spaced from, or located proximal of, the stent graft and no portion of the stent graft remains within the primary funnel 714.

Step 3 of the process 800 comprises moving the primary funnel 714 further in a proximal direction “A”, while secondary funnel 716 remains stationary, to expose at least a portion of the primary sheath 708 to provide for a grip point(s), at a location distal to distal grip 710. The amount of primary sheath 708 exposed can vary, e.g. approximately 4 inches or more, so that a user is able to manually engage the primary sheath 708 with at least one hand. The user also unscrews the turning knob 712 to create a maximum gap between the grip point and the distal grip 712.

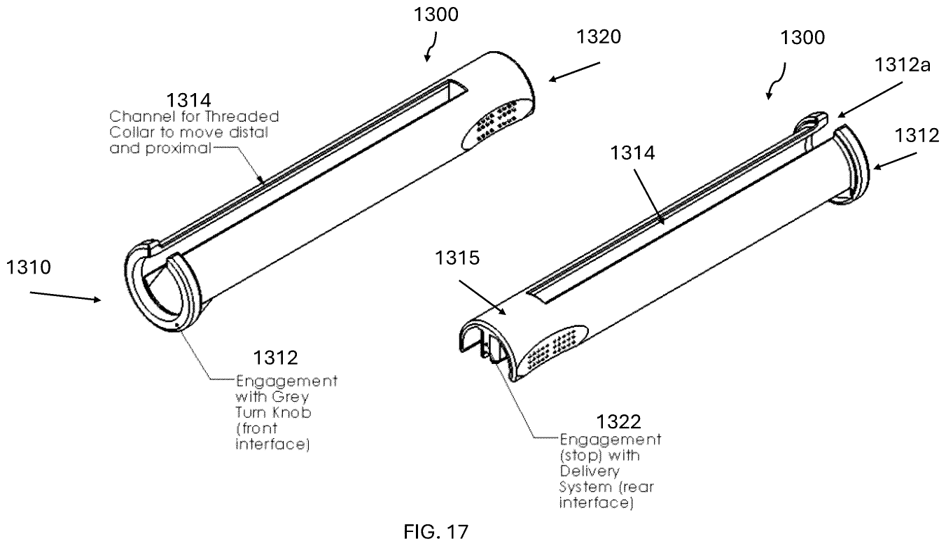

Step 4 of the process 800 comprises moving the primary sheath 708 and the turning knob 712 together, distally in the direction “B” as shown by the user hands in FIG. 8B. This separates and/or increases the space between the two funnels 714, 716. This step may be performed by multiple users, for example by two clinicians prior to deployment. Here, the distal grip 710 and the tip of the device 700 remains stationary while the primary sheath 708 and the turning knob 712 are moved together back over the stent graft 704 in the direction of arrow “B”. Additionally, the step shown in FIG. 8B can be performed with the addition of the reloading stop (see FIG. 17 1300) wherein the second user now turns the turning knob 712 in the opposite direction of the arrow shown in FIG. 18 to abut against the reloading stop causing the collar/sheath to move back over the graft, thereby recompacting or for reloading.

Step 5 of the process 800 comprises moving both the primary funnel 714 and the secondary funnel 716, once the primary sheath 708 is seated against the sheath tip 706. The two funnels can be readily removed (distally) from the device as the outer diameter of the sheath (which now also includes the modified stent graft therein) is smaller than the inner diameter of both funnels. Thus, the funnels disclosed herein allow the stent graft to be reduced in size (for reinsertion within the sheath 708) without wrapping or other torsional forces applied to the stent graft. This is advantageous in that it inhibits/prevents risk of damage to the stent graft.

In step 6, the diagram illustrates a fully reloaded step, with the modified stent graft completely retained within the sheath 708, and the distal end of the sheath 708 located at the sheath tip 706.

FIG. 9 illustrates a schematic of an exemplary graft reloading kit 400. In the illustrated embodiment, the kit 400 comprises a primary funnel 414, two loading sheaths 409a, 409b, and a secondary (smaller diameter) funnel 416. Each of these items can be formed as discrete and separate components that can be assembled together (e.g. primary funnel 414 can be threadably, or press fit, attached to loading sheath 409a; and secondary Terumo funnel 416 can be threadably attached to loading sheath 409b). The diameter of loading sheath 409a, which is connected to the primary funnel 414, is larger than the diameter of the loading sheath 409b, which is connected to the primary funnel 414.

Each loading sheath 409 is used to load and move a respective funnel. In some embodiments the loading sheaths are detachable from the funnel cone. The diameters of the two loading sheaths are incrementally different from one another, allowing for a controlled step-wise reduction in diameter of the stent graft. The diameter of 409 compresses the graft to a point in which the graft is smaller than the ID of the primary sheath/tip 708/706. This allows for 708/706 to be moved back overt the compressed graft.

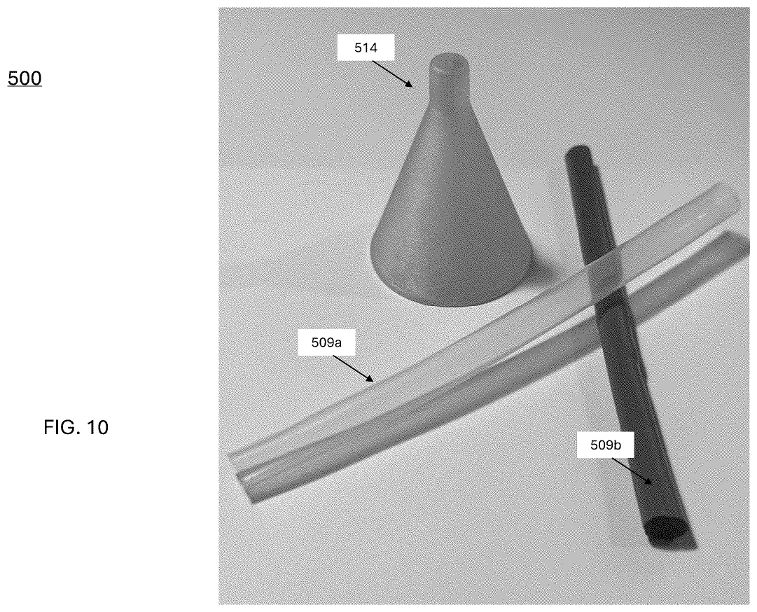

FIG. 10 is a schematic of an exemplary graft reloading kit 500. In the illustrated embodiment, the kit 500 comprises a primary funnel 514 and two loading sheaths 509a,b. Each loading sheath 509a,b is used to load and move the primary funnel 514. The diameter of loading sheath 509a, which is connected to the primary funnel 514, is larger than the diameter of the loading sheath 509b. The loading sheaths 509a,b, and/or the funnel(s), can be formed of a transparent material (e.g. Teflon) to provide visibility to the stent graft loaded therein. As shown in FIG. 10, in some embodiments only a single funnel 514 is provided for compressing the modified stent graft from an expanded to collapsed configuration for reinsertion within sheath 708.

Accordingly, recitation to a “funnel” here is not limited to a frustoconical design with a gradual taper. While it is advantageous to employ such a funnel for the primary funnel (which reduces the modified stent graft from its largest size to an intermediate expansion diameter), as this minimizes risk of damage to the modified stent graft; the secondary “funnel” 509b can be a generally elongated tubular shape. This secondary “funnel” 509b can have a tapered geometry to constrain/compress the stent graft upon insertion therein. In some embodiments, the secondary “funnel” 509b can have a split along a proximal end of the tube (e.g. approximately 10-20% of the tube 509b length) to permit deflection/expansion of the inner diameter at the end receiving the stent graft. As the tube 509b is advanced over the stent graft, once the slit terminates, the stent graft is compressed within the reminder of tube 509b. Additionally, in some embodiments, the secondary funnel is not employed by itself since it would difficult (and risk damage to the stent graft) to compress the graft down by hand to the inlet diameter of the secondary funnel. In accordance with an aspect of the present disclosure, the primary funnel and tube make the dramatic reduction in outer diameter of the stent graft then the secondary funnel and tube finishes the compaction.

### Re-Insertion of Modified Stent Graft Into Deployment Device With Sleeve as Compression Device

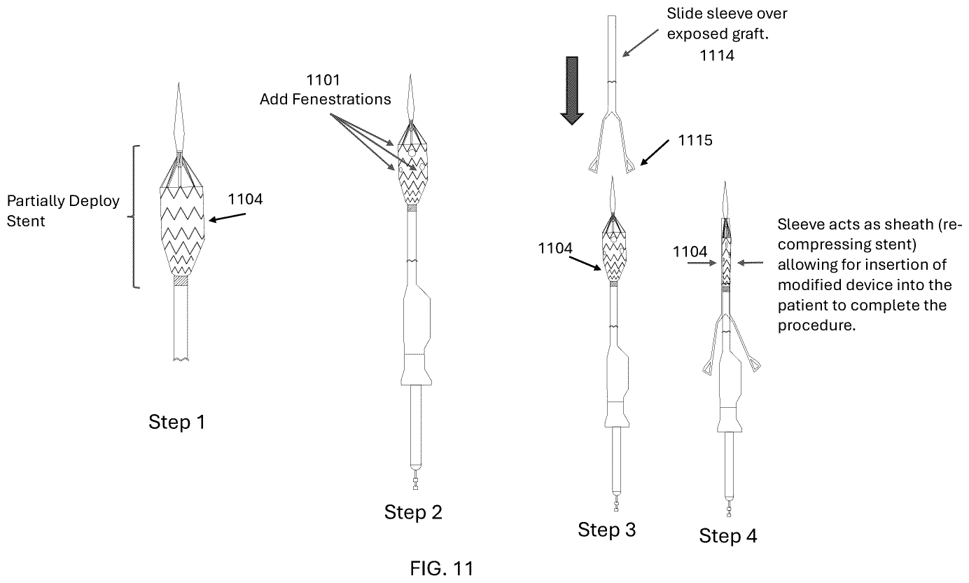

Additionally, or alternatively to the funnel(s) described above in connection with FIGS. 8-10, the modified stent graft can be reduced in size (diameter) for reinsertion via a sleeve, an exemplary embodiment of which is shown in FIGS. 11-13. As shown in Step 1 of FIG. 11, the generic (non-fenestrated) stent graft 1104 is exposed, with fenestrations 1101 added at Step 2. The fenestrations can be patient-specific and applied after the stent graft is at least partially exposed from the delivery sheath. A template, as described in connection with FIGS. 7A-7C can be employed in combination with the sleeve depicted in FIG. 11, to impart fenestrations into the at least partially deployed stent graft. A sleeve 1114 can then be advanced over the exposed stent graft 1104, with the sleeve having a larger opening or mouth at the proximal end to receive the expanded stent graft, and a narrowing diameter along the sleeve length to compress the stent within the sleeve. The sleeve can be formed from a variety of suitable materials, including fabric or plastic. In some embodiments the sleeve can be configured with elongated tethers 1115 or handles at the opening which serve as handles for an operator to grip and pull the sleeve proximally to compress the stent graft 1114. As shown, the sleeve 1114 acts as a sheath to recompress the stent graft, thereby allowing for insertion of the, now modified stent graft 1104, into a patient.

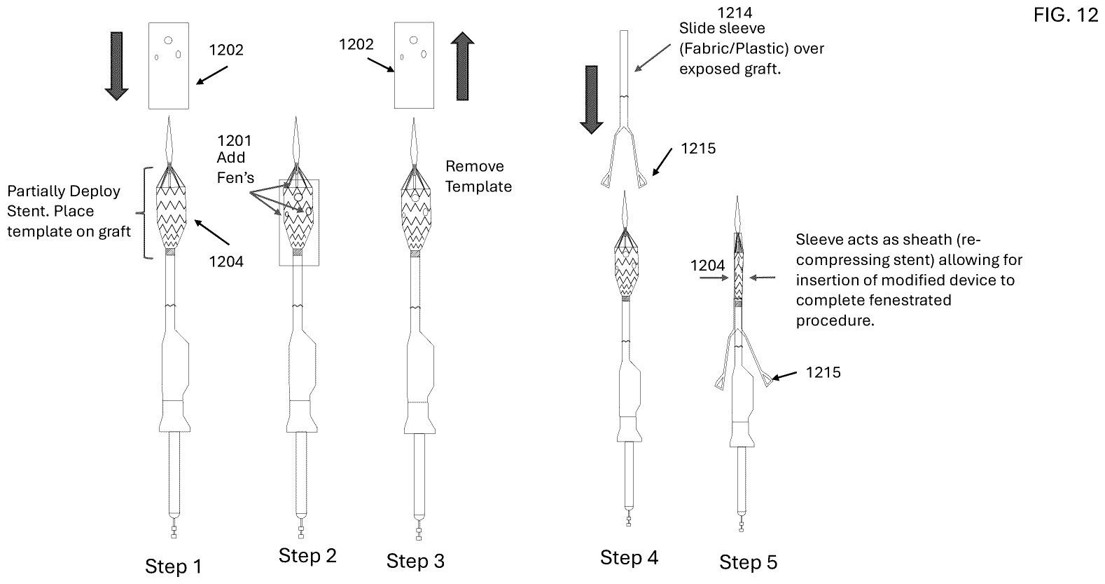

FIG. 12 depicts a similar process to the embodiment described in connection with FIG. 11, and includes a patient-specific template 1202 (as described in connection with the embodiments of FIGS. 8-10) to guide the formation of the fenestrations within the stent graft 1204.

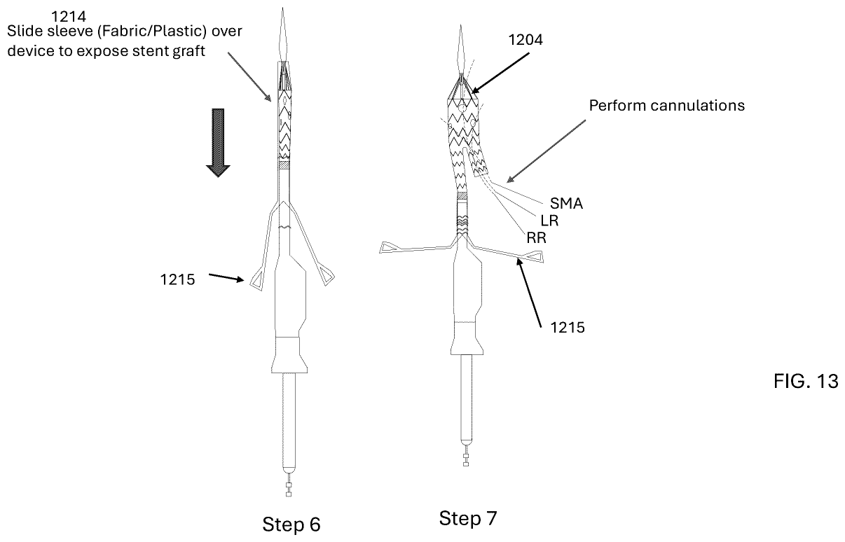

FIG. 13 illustrates additional steps that can be performed once the modified stent graft 1204 is compressed, with the sleeve 1214 continuously pulled in the proximal direction to expose a distal portion of the modified stent graft 1204. This allows for further modifications to be performed on the stent graft, e.g. cannulations to the superior mesenteric artery (SMA), etc. In some embodiments, tethers/handles 1215 remain on the delivery system throughout the entire stent graft reloading procedure, through delivery into the patient. In such embodiments, the tethers/handles 1215 serve as the actual delivery sheath itself (instead of pushing the stent graft back into the sheath of the original delivery system). The sleeve 1214 is tracked through the patient's anatomy as shown in Step 6 of FIG. 13, and deployed as shown in Step 7 (with the physician retracting the sleeve 1214 to expose the bifurcated stent graft and performing the desired cannulations).

### Reloading Stop for Insertion of Modified Stent Graft Into Deployment Device

In accordance with another aspect of the disclosure, after a stent graft is partially (or fully) deployed and modified by the physician to add fenestration locations for treatment of aortic aneurysm that encompass or affect the visceral segment of the aorta, a reloading stop can be used to facilitate reloading or reinserting the modified stent graft into the delivery device for insertion within the patient.

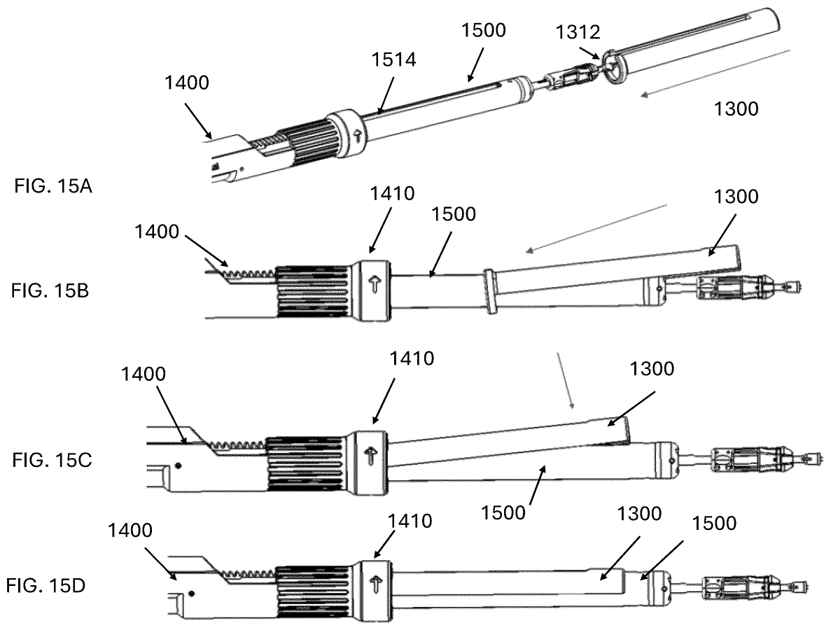

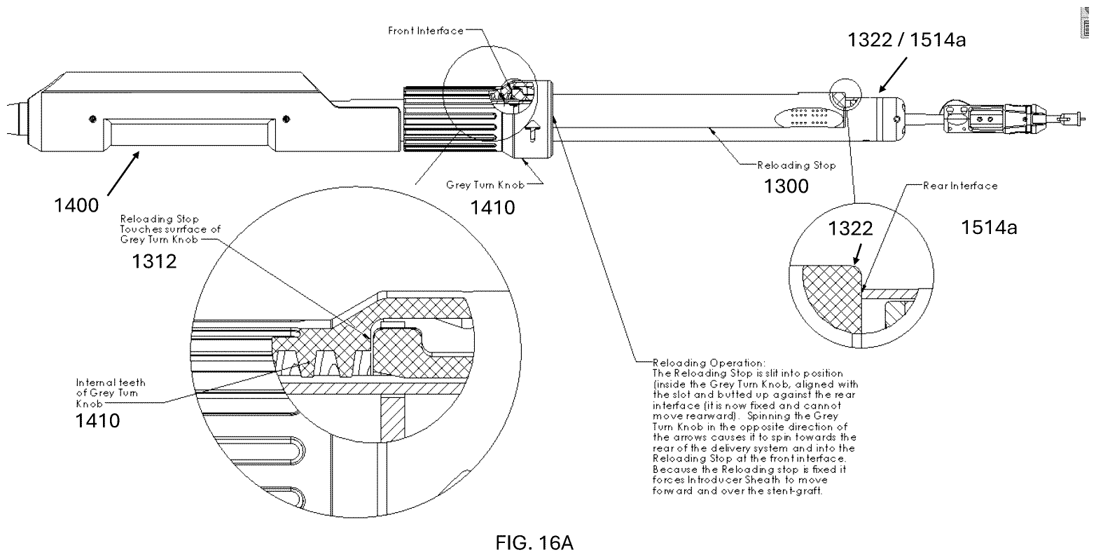

An exemplary embodiment of a reloading stop is shown in FIGS. 15-17. The reloading stop can be included as part of a kit including the patient-specific fenestration template, fenestration rings and sutures, as described above, with which a physician can impart patient-specific fenestrations into a previously assembled generic stent graft.

The reloading stop 1300 allows for the mechanical advantage of the turning knob and lead screw to retract the sheath so that the secondary user (e.g. second pair of hands in FIG. 8B) is not burdened with moving the sheath into position. In the absence of the reloading stop 1300, the physician can impart patient-specific fenestrations and then manually advance the sheath over the now fenestrated stent graft to reload the stent graft into the delivery system for subsequent deployment in the patient. This manual operation, in the absence of the reloading stop 1300, can require a significant amount of indiscriminate force and increase the risk of damaging the stent graft and delivery system during reloading. Due to the variable nature of the resistance of the system to being reloaded, manual operation in the absence of the reloading stop 1300 can lead to abrupt changes to the speed of reloading if a physician or their assistant are applying too much force at a particular time, thus increasing the chances of damaging the stent graft or delivery system.

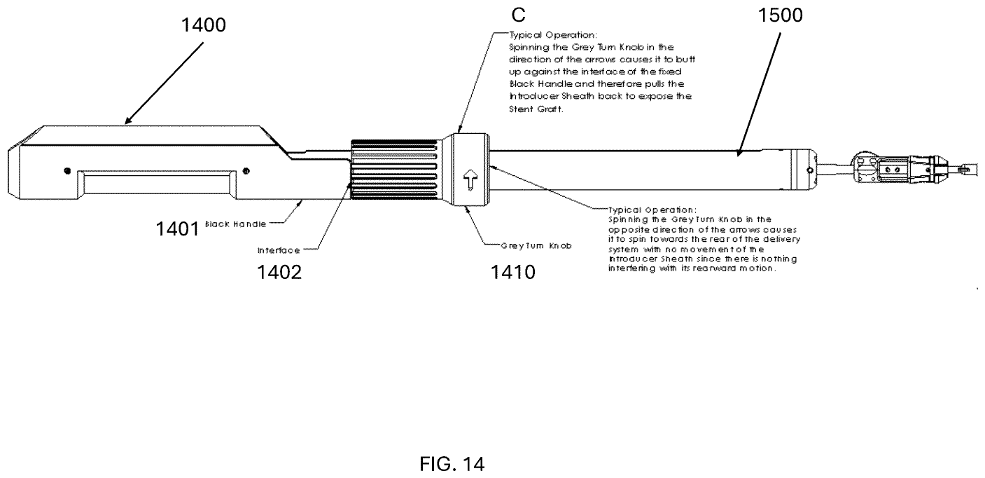

An exemplary embodiment of such a manual stent graft reloading system is shown in FIG. 14. A physician holds handle 1400 and rotates knob 1410 in a clockwise direction (as shown by arrow “C”), which (initially) displaces a distal portion 1402 of the handle against a proximal portion of the handle 1401. The portion 1402 of the handle is fixed in place with no displacement along the longitudinal axis. When distal portion 1402 abuts the interface of proximal portion 1401 there is no further displacement of knob portion 1410 permissible. Continued rotation of knob 1410 engages the sheath to displace or retract the sheath in the proximal direction and thereby expose the stent graft (for incorporating patient-specific modifications, e.g. fenestrations).

However, counterclockwise rotation of the knob 1410 does not advance the sheath distally to cover or reload the stent graft in the embodiment shown in FIG. 14. Rather, counterclockwise rotation of the knob 1410 only displaces the knob 1410 itself in the distal direction (while the sheath remains in its retracted, proximal position). That is, knob 1410 is free to move along the longitudinal axis since there is no obstruction or blocking structure in its path.

Thus, in accordance with another aspect of the disclosure, the reloading stop 1300 can adapt a stent graft delivery system, such as the system of FIG. 14, which is initially designed to only be used to deploy the stent-graft (i.e. pull the introducer sheath proximally towards the handle to expose the stent-graft) into a dual purpose or dual mode of delivery actuation system. That is, with the addition of the reloading stop 1300, the delivery system can also operate to push the introducer sheath distally towards the tip and cover the stent-graft. This provides a consistent and efficient stent graft reloading process. By providing the mechanical advantage of the turning knob, the action of “pushing” the sheath back over the stent graft can be done in a significantly more controlled manner than without the reloading stop 1300. Using the reloading stop 1300, the effort required to reload the sheath will be much less, and most importantly the chances of damaging the stent graft or delivery system are much lower. The same actuator mechanism can be used for both retracting and advancing the sheath over the stent graft. In the exemplary embodiment depicted, the actuation mechanism is a rotatable knob 1410, which can rotate clockwise and counterclockwise (relative to the longitudinal axis extending through the handle 1400). Though artisans of ordinary skill will recognize that alternative actuator mechanisms (e.g. buttons, push and/or spring operated) are also within the scope of this disclosure.

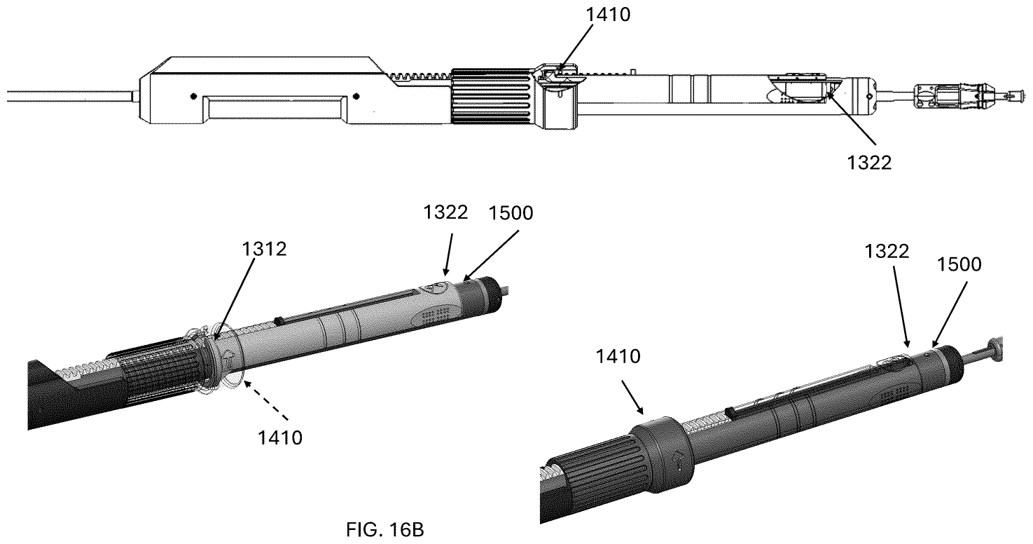

Referring to FIGS. 15-16 (which shows the insertion of the reloading stop 1300 into a delivery system) and FIG. 17 (which shows the reloading stop 1300 in isolation), the reloading stop 1300 has a first interface 1310 on the proximal end which is designed to engage with the rotatable knob 1410, and a second interface 1320 on the distal end which is designed to engage with the elongated shaft of the delivery device.

The first interface 1310 on the proximal end of the reloading stop 1310 can include a rim 1312 with a larger diameter from the remainder of the longitudinally extending reloading stop, such that the rim 1312 extends laterally outward from the remainder of the reloading stop 1300. The inner diameter of the rim 1312 is sized to fit over the outer diameter of the longitudinal shaft 1500. while the outer diameter of the rim 1312 is sized to fit under the inner diameter of the turning knob 1410, as shown in FIGS. 15A-15D. Reloading stop 1300 also includes a slit or channel 1314 extending along the longitudinal axis from the rim 1312 to a location spaced from the second interface 1320. This geometry (e.g. solid portion 1315) provides additional structural integrity of the reloading stop as this portion of the reloading stop abuts the handle and creates the locking point for the reloading so that it will butt up against the turning knob 1410. The channel 1314 is sized to be long enough to ensure the stent-graft can be exposed enough to place fenestrations in the desired location. If the channel 1314 is too short the threaded collar (black rack) will hit it before enough surface area of the stent-graft is exposed.

In the exemplary embodiment shown, channel 1314 is formed through the rim 1312 and extends approximately 70%-90% of the length of the reloading stop 1310. Thus, the rim 1312 does not form a completely closed circular structure, but rather an opened U-shape structure. The opening 1312a provides structural flexibility permitting the proximal end of the reloading stop to deform during insertion into the rotatable knob 1410, thereby reducing risk of damage to either component.

The reloading stop 1300 can be formed in a generally tubular shape and sized to fit within the longitudinal shaft of the deployment device, with channel 1314 configured to receive a threaded collar and permit displacement of the collar in the proximal and distal directions. Additionally or alternatively, in some embodiments, the reloading stop 1300 can be snap-fit around a portion of the longitudinal shaft of the deployment device.

The reloading stop 1300 can include an internal protrusion 1322, e.g. extending normal to the interior surface of the reloading stop, at the distal end 1320 or rear/second interface. This internal protrusion 1322 serves as a structural barrier that engages an interface of the delivery shaft to prohibit longitudinal displacement of the reloading stop 1300, as described in further detail below. Internal protrusion 1322 can be formed as a single protrusion (e.g. a “T” shaped structure) or multiple protrusions (e.g. an “H” shaped structure). The protrusion 1322 is designed to set on the hypotube that runs through the delivery system. In some embodiments, the protrusion 1322 sits on top of the handle assembly and nests into the handle body to create a new engagement point for the turn knob 1410 so the mechanical advantage can be utilized.

As shown in the various stages of FIGS. 15A-D, reloading stop 1300 can be coupled to the delivery shaft 1500 by aligning the two components along the longitudinal axis, as shown in FIG. 15A, and advancing the reloading stop 1300 in the proximal direction to bring rim 1312 towards the knob 1410. As shown in FIG. 15B, reloading stop 1300 can be pitched or tilted such that proximal end with rim 1312 is lower than distal end 1322, so that the bulbous rim (having a larger diameter than the remainder of the reloading stop 1310) can be received within / under the knob 1410. In some embodiments, the rim 1312 and knob 1410 can be sized such that an interference fit is formed between the two components, and optionally a signal (e.g. tactile, haptic, audible) is provided to confirm proper engagement of the components.

Once the rim 1312 and knob 1410 are coupled, the distal portion of the reloading stop 1300 can be lowered to engage the shaft 1500 of the delivery device, as shown in FIG. 15C. The internal protrusion 1322 is sized and positioned to be received within a slot 1514 of the shaft 1500 of the delivery device, with the distal edge of the protrusion 1322 engaging the endpoint of slot 1514a of the delivery device shaft, as shown in FIG. 15D and FIG. 16A-B. Thus, the protrusion 1322 abuts or blocks longitudinal movement of the reloading stop 1300, maintaining the reloading stop in a longitudinally fixed position.

Once the reloading stop is inserted within the delivery system, as shown in FIG. 16, counterclockwise rotation of knob 1410 displaces the distal portion of handle 1402 (as described above in connection with FIG. 14) distally into engagement with the reloading stop proximal end 1310 which prohibits further displacement of distal portion of handle 1402. Continued counterclockwise rotation of knob 1410 then engages the sheath to displace the sheath distally and re-cover the (now fenestrated) stent graft. The reloading stop 1300 can be employed with the funnels (FIGS. 8-10) and/or sleeve (FIGS. 12-13) as described above, and can be coupled to the delivery shaft before, or after, the funnels or sleeves are employed. In some embodiments, the reloading stop 1300 can be inserted at anytime as long as the stent-graft is partially deployed. The reloading stop 1300 is positioned on the back end of the device, away from the introducer sheath and funnel placement location(s).

Reloading stop 1300 can be removed from the delivery system, if so desired, by a reverse order of operations of FIG. 15D-Fig. 15A, with the distal end being raised to remove the protrusion 1322 from within channel 1514 of the delivery device shaft. Thereafter, the rim 1312 can be disengaged or decoupled from the knob 1410 and the reloading stop 1300 removed from the delivery system. Thus, the reloading stop 1300 facilitates the ease of reloading a stent-graft and reduces the potential for stent-graft or delivery system damage.

Similarly to the kit disclosed above in connection with FIGS. 5G-H for forming patient-specific fenestration(s) to a universal stent graft (i.e. no fenestrations initially included), the kit, as shown in FIG. 19, can further include:
iv) a patient-specific template 500 (which can be removably coupled to the stent graft once exposed from the sheath);
v) at least one fenestration ring 550 (which can be attached to the fenestration edge/boundary)
vi) at least one suture 560 (which can be a closed loop, or open thread and used to attach the fenestration ring to the stent graft);
vii) Funnels 400 and/or sleeves 1214 (for compressing the fenestrated stent graft to facilitate reloading within the deployment device).

Each component can be separately packaged to ensure sterility, and the reloading stop 1300 can be provided separately.

While the disclosed subject matter is described herein in terms of certain preferred embodiments, those skilled in the art will recognize that various modifications and improvements may be made to the disclosed subject matter without departing from the scope thereof. Moreover, although individual features of one embodiment of the disclosed subject matter may be discussed herein or shown in the drawings of the one embodiment and not in other embodiments, it should be apparent that individual features of one embodiment may be combined with one or more features of another embodiment or features from a plurality of embodiments.

In addition to the specific embodiments claimed below, the disclosed subject matter is also directed to other embodiments having any other possible combination of the dependent features claimed below and those disclosed above. As such, the particular features presented in the dependent claims and disclosed above can be combined with each other in other manners within the scope of the disclosed subject matter such that the disclosed subject matter should be recognized as also specifically directed to other embodiments having any other possible combinations. Thus, the foregoing description of specific embodiments of the disclosed subject matter has been presented for purposes of illustration and description. It is not intended to be exhaustive or to limit the disclosed subject matter to those embodiments disclosed.

It will be apparent to those skilled in the art that various modifications and variations can be made in the method and system of the disclosed subject matter without departing from the spirit or scope of the disclosed subject matter. Thus, it is intended that the disclosed subject matter include modifications and variations that are within the scope of the appended claims and their equivalents.

What is claimed:

1. A system for a reloading a medical device comprising:
an elongated shaft having a proximal end and a distal end defining a longitudinal axis therebetween, the elongated shaft including a sheath configured to retain a medical device therein;
a handle;
a rotatable knob coupled to the sheath, the rotatable knob configured to displace the sheath in a first direction when the rotatable knob rotates in a first direction, and to displace the sheath in a second direction when the rotatable knob rotates in a second direction;
a reloading stop, the reloading stop having a first interface disposed on a proximal end and a second interface disposed on a distal end thereof;
wherein the first interface of the reloading stop is configured to engage one of the rotatable knob and the elongated shaft, and the second interface of the reloading stop is configured to engage the other one of the rotatable knob and elongated shaft.

2. The system of claim 1, wherein the first direction is clockwise, and the second direction is counterclockwise.

3. The system of claim 1, wherein the first direction is counterclockwise, and the second direction is clockwise.

4. The system of claim 1, wherein the reloading stop includes a channel disposed along a longitudinal axis thereof.

5. The system of claim 1, wherein the channel extends from a proximal end to a location spaced from the distal end of the reloading stop.

6. The system of claim 4, wherein the channel is configured to receive a threaded collar therein, with the threaded collar configured for displacement in the proximal and distal directions.

7. The system of claim 1, wherein the reloading stop includes a rim disposed at a proximal end, the rim having a diameter larger than the reminder of the reloading stop.

8. The system of claim 7, wherein the rim is disposed within the rotatable knob.

9. The system of claim 1, wherein the reloading stop includes an internal protrusion disposed at a distal end, the internal protrusion extending normal to the interior surface of the reloading stop.

10. The system of claim 1, wherein the reloading stop is coupled to the elongated shaft in a longitudinally-fixed position.

11. The system of claim 1, wherein the reloading stop is removable from the elongated shaft.

12. The system of claim 1, wherein rotation of the rotatable knob in the first direction displaces the sheath in a distal direction to expose the medical device, and rotation of the rotatable knob in the second direction displaces the sheath in a proximal direction to cover the medical device.

13. The system of claim 12, wherein the fist direction is clockwise, and the second direction is counterclockwise.

14. The system of claim 12, wherein the fist direction is counterclockwise, and the second direction is clockwise.
